(19) United States
(12) Patent Application Publication
(10) Pub. No.: US 2005/0119613 A1
(43) Pub. Date: Jun. 2, 2005

(54) Fluid delivery trocar-cannula complex, fluid delivery accessory, and method for delivering fluids during minimally invasive surgery

(76) Inventors: Stephen P. Moenning, Punta Gorda, FL (US); Richard L. Grant, Cincinnati, OH (US); Kevyn M. Irving, Mason, OH (US)

Correspondence Address: WOOD, HERRON & EVANS, LLP, 2700 CAREW TOWER, 441 VINE STREET, CINCINNATI, OH, 45202, US

(21) Appl. No.: 11/007,410

(22) Filed: Dec. 8, 2004

Related U.S. Application Data
(63) Continuation-in-part of application No. 10/786,647, filed on Feb. 25, 2004.
Application No. 10/786,647 is a continuation of application No. PCT/US02/29356, filed on Sep. 17, 2002.
Application No. 10/786,647 is a continuation-in-part of application No. 09/934,399, filed on Aug. 21, 2001, now Pat. No. 6,695,815.
Application No. 09/934,399 is a continuation of application No. 09/511,100, filed on Feb. 23, 2000, now Pat. No. 6,302,873.
(60) Provisional application No. 60/325,806, filed on Sep. 28, 2001.
(60) Provisional application No. 60/341,032, filed on Dec. 12, 2001.
(60) Provisional application No. 60/552,048, filed on Mar. 10, 2004.

(51) Int. Cl. A61M 31/00

(52) U.S. Cl. 604/93.01; 604/264

(57) ABSTRACT

A medical apparatus for dispensing a biologically active liquid includes a trocar-cannula complex configured to be inserted through selected tissue of a patient and including a cannula and an elongated trocar having a closed converging tip at a distal end thereof and a fluid delivery channel. The cannula includes a working channel through which medical instruments may be advanced. A fluid delivery device may be removably coupled to the cannula. The fluid delivery device includes an inner portion adapted to receive the biologically active liquid, an outer surface adapted to contact the tissue of the patient, and at least one pathway from the inner portion to the outer surface for delivering the biologically active liquid from the inner portion to the outer surface when the outer surface is in contact with the tissue.

The present application is a continuation-in-part of U.S. patent application Ser. No. 10/786,647, filed on Feb. 25, 2004 (pending), which is a continuation of PCT Serial No. PCT/US02/29356 filed on Sep. 17, 2002 (expired) which claims the priority of U.S. Provisional Patent Application Ser. No. 60/325,806, filed on Sep. 28, 2001 (now abandoned) and U.S. Ser. No. 60/341,032, filed on Dec. 12, 2001 (now abandoned), and which is a continuation-in-part of U.S. Ser. No. 09/934,399, filed on Aug. 21, 2001 (now U.S. Pat. No. 6,695,815) which is a continuation of U.S. Ser. No. 09/511,100 filed on Feb. 23, 2000 (now U.S. Pat. No. 6,302,873). The present application is also related to U.S. Provisional Application Ser. No. 60/552,048, filed on Mar. 10, 2004 (pending). The disclosures of each of these prior related applications are hereby fully incorporated by reference herein.

### FIELD OF THE INVENTION

This invention generally relates to trocar assemblies and related accessories and, more specifically, to devices used to irrigate a port site during minimally invasive surgery.

### BACKGROUND OF THE INVENTION

Minimally invasive surgery is a popular alternative to more traditional surgery. This is due to the fact that minimally invasive surgery generally results in less pain and shorter hospital stays for the patient. Also, the cost of performing a surgical procedure through minimally invasive techniques can be substantially less than more traditional surgical approaches.

Minimally invasive surgical techniques require access into the body of a patient through a small working channel of an apparatus known as a trocar-cannula complex or assembly. A relatively small access incision is made in the patient at the appropriate location to receive the trocar-cannula complex. When the trocar-cannula complex is combined with long, narrow instruments, the resulting assembly allows a surgeon to work inside the body through the small access incision or port site. This approach has resulted in the aforementioned clinical advantages and extensive health care cost savings.

Traditionally, the trocar-cannula complex has been configured with three parts. The first part is the top portion and is referred to in the medical industry as the hub. The hub defines the entrance to the trocar-cannula complex and also includes various seals and air insufflation components. The second part is the trocar, which is a long, narrow blade or closed, converging tip extendable through a cannula to allow smooth penetration into the body of the patient through the tissue layers. The third portion is an outer cannula which is a tubular member of the complex adapted to pass into the body cavity. The outer cannula provides an interface between the patient's tissue at the access incision or port site and the trocar assembly.

Minimally invasive surgery has grown in popularity in recent years and many new types of trocar-cannula products have been proposed and introduced to address different surgical needs and procedures. The various trocar-cannula complexes include reusable and disposable cannulas and trocars, as well as hybrid varieties that comprise combinations of reusable and disposable components of the trocar-cannula complexes. A complex which is a combination of reusable and disposable components is known as a resposable device. Such devices continue to improve surgical outcomes and economics.

Animal studies on cancer treatments involving the performance of minimally invasive surgery point to a growing body of evidence which supports the concept of delivering an irrigant to the port site before, during or after the surgical procedure. In order for surgeons to continue with a minimally invasive procedure, such as a laparoscopic, arthroscopic, or thoracoscopic case, the cannula must stay in the port site of the patient. Thus, the irrigants were delivered by a syringe and needle and included substances such as betadine, saline and lidocaine. These studies showed that irrigating the port site with such substances immediately after the surgical procedure beneficially resulted in a lower incidence of infection or less pain, depending on the irrigant. However, the technique also resulted in increased operative time and increased exposure of the surgical staff to needle sticks. In addition, the potential for contaminants to spread to the port site during the surgery has been well documented. Irrigation performed only at the end of the surgical procedure unfortunately cannot reduce patient exposure to contaminants during the procedure.

The above-incorporated patents and patent applications disclose various manners of delivering irrigants to a port site.

There is a need for even more effective and efficient delivery of fluids to an access point or port in the body of a patient before, during, and/or after the performance of minimally invasive surgery. Such delivery of fluid(s) could assist in patient treatment, such as through the delivery of cancer treatment medication or other medication, as well as reduction of port site contamination and infection, and reduction of post-operative pain. Other uses of the invention may be made in connection with delivering any desired fluid to a patient.

### SUMMARY OF THE INVENTION

The present invention generally relates to a medical apparatus for dispensing a biologically active fluid or liquid on the outside of a cannula. The apparatus includes a fluid delivery device which is configured to be removable coupled to the cannula and includes an inner portion adapted to receive the biologically active liquid and an outer surface adapted to contact any selected tissue of the patient. At least one pathway extends from the inner portion to the outer surface for delivering the biologically active liquid to the outer surface when the outer surface is in contact with the tissue.

Various embodiments are disclosed in which, for example, the inner portion of the fluid delivery device may further comprise a sponge material, a void space, multiple discrete void spaces, etc., all of which serve to convey the liquid to the outer surface of the device at an appropriate time, either immediately upon insertion of the trocar-cannula complex into the port site of the patient, or at some desired time or times after insertion of the trocar-cannula complex. As additional examples, the plurality of discrete void spaces may further comprise a plurality of fluid delivery channels extending generally along the length of the cannula. For example, these channels may be parallel to the length of the cannula or may spiral along the length of the cannula, or be formed in any other configuration. The fluid delivery device may be removably engaged with the cannula in many different manners, such as through frictional engagement or any other type of mechanical engagement or adhesive engagement.

An actuator may be coupled to the fluid delivery device and configured to force the biologically active liquid along the pathway to the outer surface into contact with the tissue of the patient. The actuator, for example, may comprise a pump mechanism of many different types. For example, the actuator may comprise a syringe, or another type of movable piston-type pump, or screw-type pump. In another embodiment, the pump mechanism comprises one or more flexible members similar to primer mechanisms which may be depressed by the user to displace fluid from the inner portion along the pathway to the tissue of the patient. The fluid delivery device may include more than one actuator and more than one type of actuator. For example, a preselected amount of the liquid may be initially contained in the fluid delivery device and delivered to the patient via one pump mechanism and, if necessary, a second pump mechanism such as a syringe may deliver an additional amount of the same liquid or a different liquid, for example, if the first stored amount of liquid is depleted during the surgical procedure.

In accordance with another aspect of the invention, a fluid path selection device may be operatively coupled with the fluid delivery device for selectively allowing the biologically active liquid to be dispensed along different portions of the length of the fluid delivery device. For example, in one position fluid may be dispensed into one or more channels which release the liquid along a first 1cm length portion of the fluid delivery device, and in a second position the fluid is released along a second 1 cm length portion of the fluid delivery device. Alternatively, or in addition, the fluid path selection device may be adjusted to deliver fluid along preselected shorter or longer extents of the fluid delivery device. This type of adjustability in the fluid exits allows, for example, for fluid to be accurately delivered to an appropriate location depending on the tissue requirements of the patient. Typically, this will mean delivering the fluid to the tissue between the skin of the patient and an underlying body cavity or space in which a procedure is undertaken.

A method of performing a minimally invasive surgical procedure in accordance with the invention generally involves affixing the removable fluid delivery device to the cannula portion of a trocar-cannula complex, and introducing the trocar-cannula complex and the fluid delivery device through a port site of a patient during, for example, laparoscopic, arthroscopic, or thoracoscopic cases. Fluid is then delivered from a fluid passage in the fluid delivery device to the outside surface thereof and into contact with the tissue of the patient within the port site. Various biologically active liquids may be delivered in this manner, including irrigants, pain medication, tissue adhesives, or any other liquid substance which is desired at the port site.

It will be understood that various advantages and additional features of the invention will become more readily apparent to those of ordinary skill upon review of the following detailed description of the preferred embodiments taken in conjunction with the accompanying drawings.

### BRIEF DESCRIPTION OF THE DRAWINGS

FIG. 1 is a disassembled perspective view of a fluid delivery device constructed in accordance with a first embodiment of the invention.

FIG. 2A is a partially sectioned perspective view of a trocar-cannula complex or assembly including the fluid delivery device of FIG. 1.

FIG. 2B is a perspective view, again partially cross sectioned and shown inserted into a patient's tissue.

FIG. 3A is a longitudinal cross sectional view of a fluid delivery device constructed in accordance with another embodiment of the invention.

FIG. 3B is a cross sectional view similar to FIG. 3A, but illustrating the assembly of a trocar-cannula complex with the fluid delivery device.

FIG. 3C is a cross sectional view similar to FIG. 3B, but illustrating the insertion of the fully assembled device into the tissue of a patient.

FIG. 4 is a longitudinal cross sectional view of another fluid delivery device constructed in accordance with the invention.

FIG. 4A is a cross sectional view taken along line 4A-4A of FIG. 4.

FIG. 5 is a partially fragmented perspective view illustrating another embodiment of a fluid delivery device in accordance with the invention.

FIG. 6 is a partially fragmented perspective view illustrating another embodiment of a fluid delivery device in accordance with the invention.

FIGS. 7A and 7B are partially cross sectioned views showing a fluid delivery device of the invention being assembled with a trocar-cannula complex.

FIG. 8 is a partially cross sectioned view of a trocar-cannula complex assembled with a fluid delivery device according to another embodiment of the invention.

FIG. 8A is an enlarged view of the distal end of the trocar-cannula complex shown in FIG. 8.

FIG. 9 is a perspective view of the fluid delivery device shown in FIGS. 8 and 8A.

FIG. 10 is a perspective view of a fluid delivery device constructed in accordance with another embodiment of the invention.

FIG. 11 is a cross sectional view illustrating the fluid delivery device of FIG. 10 assembled with a trocar-cannula complex.

FIG. 11A is an enlarged view of the proximal portion of the fluid delivery device shown in FIG. 11.

FIG. 12A is a cross sectional view taken along line 12A-12A of FIG. 11.

FIG. 12B is a cross sectional view similar to FIG. 12A, but illustrating an adjusted position for delivering fluid along a different path in the fluid delivery device.

FIG. 13 is a transverse cross sectional view of an alternative fluid delivery device illustrating another manner of manufacture.

FIG. 14 is a perspective view of a fluid delivery device constructed according to another embodiment of the invention.

FIG. 15 is a cross sectional view similar to FIG. 11A, but illustrating one type of pump mechanism for forcing liquid through the fluid delivery device.

FIG. 16A is a cross sectional view of a fluid delivery device constructed in accordance with another embodiment of the invention having a screw pump mechanism.

FIG. 16B is a cross sectional view similar to FIG. 16A, but illustrating the screw pump mechanism in a depressed position for forcing liquid out of the fluid delivery device.

FIG. 17A is a cross sectional view of a fluid delivery device in accordance with another embodiment of the invention, and including a piston pump mechanism.

FIG. 17B is a cross sectional view similar to FIG. 17A, but illustrating the piston pump mechanism in a depressed position.

FIG. 18 is a cross sectional view of a fluid delivery device in accordance with another embodiment of the invention, and illustrating a pump mechanism in the form of a flexible member adapted to be depressed by a user to displace fluid from the fluid delivery device.

FIG. 19A is a disassembled perspective view of a disposable fluid container adapted to be coupled with a fluid delivery device of the present invention.

FIG. 19B is a disassembled cross sectional view illustrating the assembly of the container shown in FIG. 19A with a fluid delivery device of the invention.

FIG. 19C is a cross sectional view similar to FIG. 19B, but illustrating the assembled condition of the container and fluid delivery device.

### DETAILED DESCRIPTION OF THE PREFERRED EMBODIMENTS

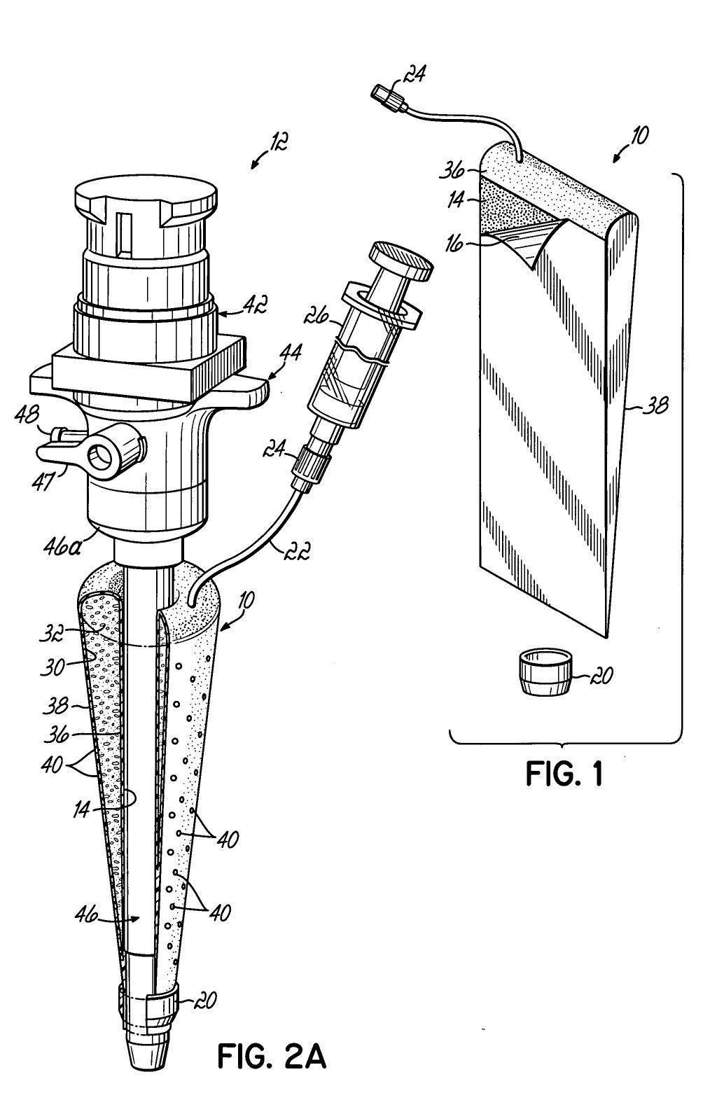

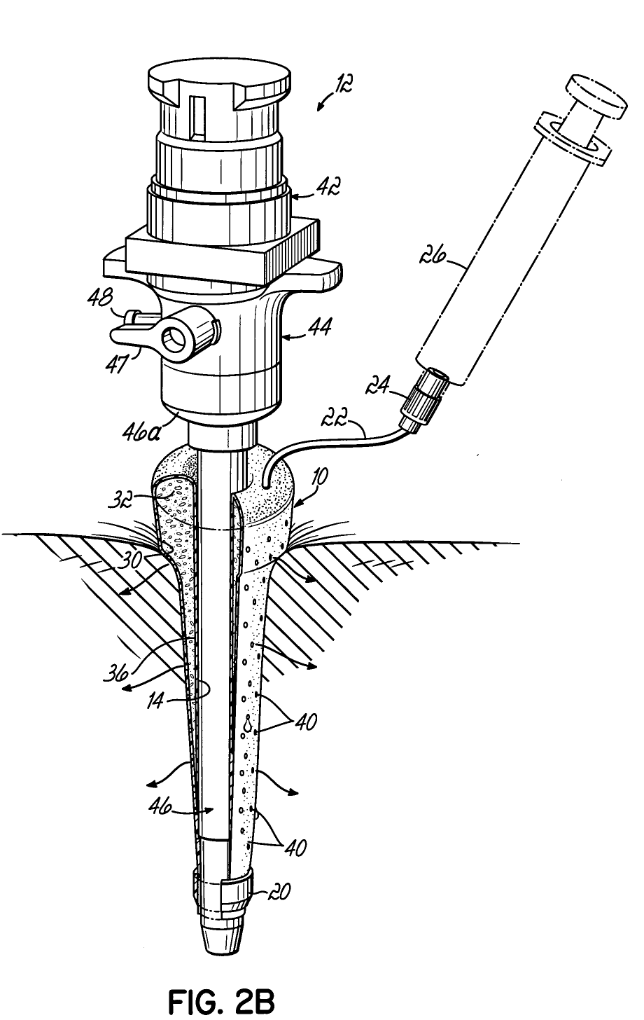

FIG. 1 illustrates a fluid delivery device 10 constructed in accordance with one embodiment of the invention before application to a trocar-cannula complex 12 as shown in FIGS. 2A and 2B. The device 10 of FIG. 1 may be formed from a sponge-type material and is shown having a thickness greater than that which would most likely be used in practice, but this is for illustrative purposes of clearly showing the layers of the device 10 in FIGS. 2A and 2B. This device 10 may have an adhesive backing 14 which may be exposed by peeling away a paper layer 16 and may also include a retaining member 20 which is slipped over the distal end of the device as shown in FIGS. 2A and 2B for helping to maintain the distal end in place, especially during insertion into the port site of a patient. As further shown in these figures, the device 10 includes a fluid delivery line 22 and a standard luer connector 24, for example, for attaching a syringe 26 The syringe 26 may be used to deliver the biologically active fluid or liquid into a space or inner portion 30 of the device which may be filled with a sponge-like absorbent material 32 encased by inner and outer skins 36, 38. The outer skin 38 may include perforations 40 for allowing the fluid to travel along a fluid path within the sponge 32 and onto the outer surface of the device 10 through the perforations 40. Although the perforations 40 are shown along substantially the entire length of the device 10, these may instead be selectively formed along a specific portion of the length of the device 10.

As further shown in FIGS. 2A and 2B, the trocar-cannula complex 12 may include a conventional trocar 42 and hub assembly 44. A cannula 46 includes a base portion 46a coupled with the hub assembly 44. The hub assembly 44 may further include an insufflation valve 47 and a gas inlet 48 for receiving a pressurized gas, such as CO₂ as is conventionally used in laparoscopic procedures. As shown in FIG. 2B, when the apparatus 10, 12 is inserted in to the port site, fluid may be delivered through the perforations 40 and into the tissue of the patient, for example, through a squeezing action on the sponge material 32 after the sponge material 32 has been saturated with the desired fluid.

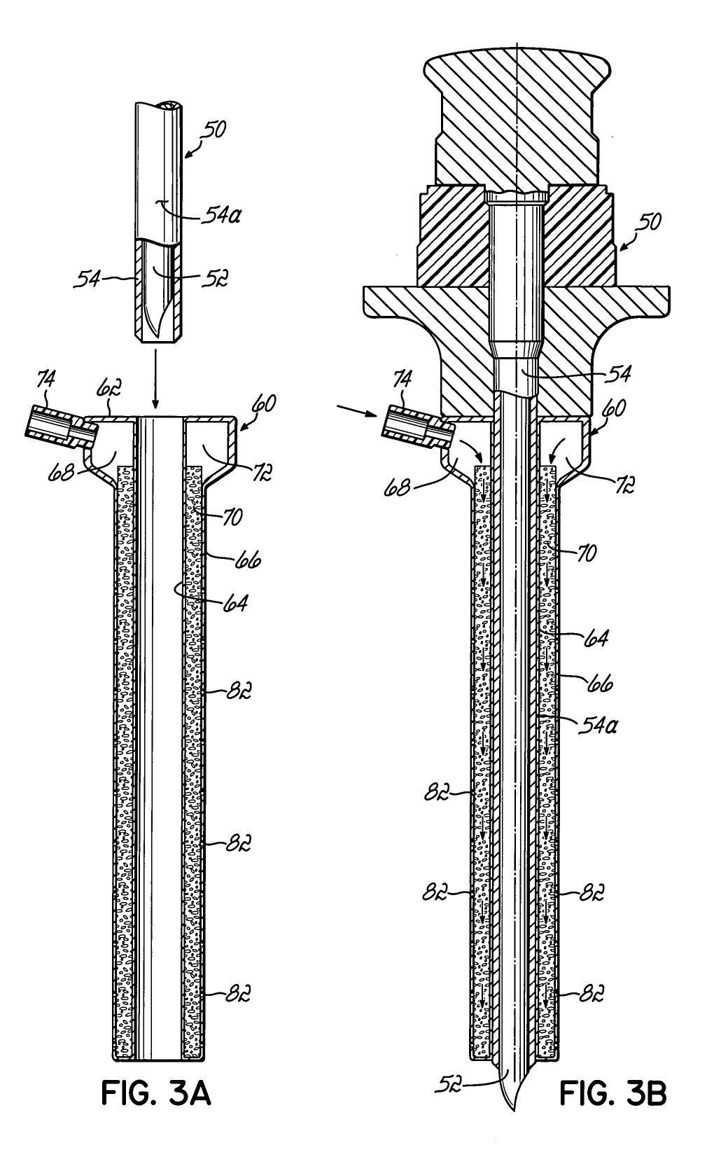

FIGS. 3A-3C illustrate another embodiment of the invention in cross section. More specifically, a trocar-cannula complex 50 includes a trocar 52 and cannula 54. The trocar-cannula complex 50 is inserted into a fluid delivery device 60 in a removable fashion and, for example, is retained on the outer surface 54a of the cannula 54 with a friction fit. The fluid delivery device 60 comprises a housing 62 having an inner wall 64 and an outer wall 66 with an essentially cylindrical, annular space 68 therebetween that receives a sponge material or other fluid absorbent material 70. A chamber or void space 72 is formed at the proximal end of the device 60 and is in fluid communication with a suitable coupling 74. The coupling 74 may be secured to a fluid delivery line or directly to a fluid actuator, such as a syringe or other type of pump mechanism (not shown). The fluid 80 will be delivered to the chamber 72 as schematically shown in FIG. 3C whereupon it will soak into and saturate the sponge or fluid absorbent material 70. The trocar-cannula complex 50 with the attached fluid delivery device 60 is preferably not inserted until full saturation of the sponge material 70 has taken place. At that point, the trocar-cannula complex 50 and fluid delivery device 60 are inserted and, as shown in FIG. 3C, at least the outer wall or skin 66 is flexible such that the sponge material 70 is compressed by the patient's tissue during insertion and the biologically active liquid is forced out of perforations 82 in the outer wall 66 into the tissue of the patient as indicated by the arrows in FIG. 3C. As with several, if not all, embodiments illustrated herein, the thickness of the fluid delivery device is exaggerated for illustrative purposes related to clearly showing certain details. Also, it may be desirable to taper the distal end of the fluid delivery device, if necessary, to aid in its insertion through a port site.

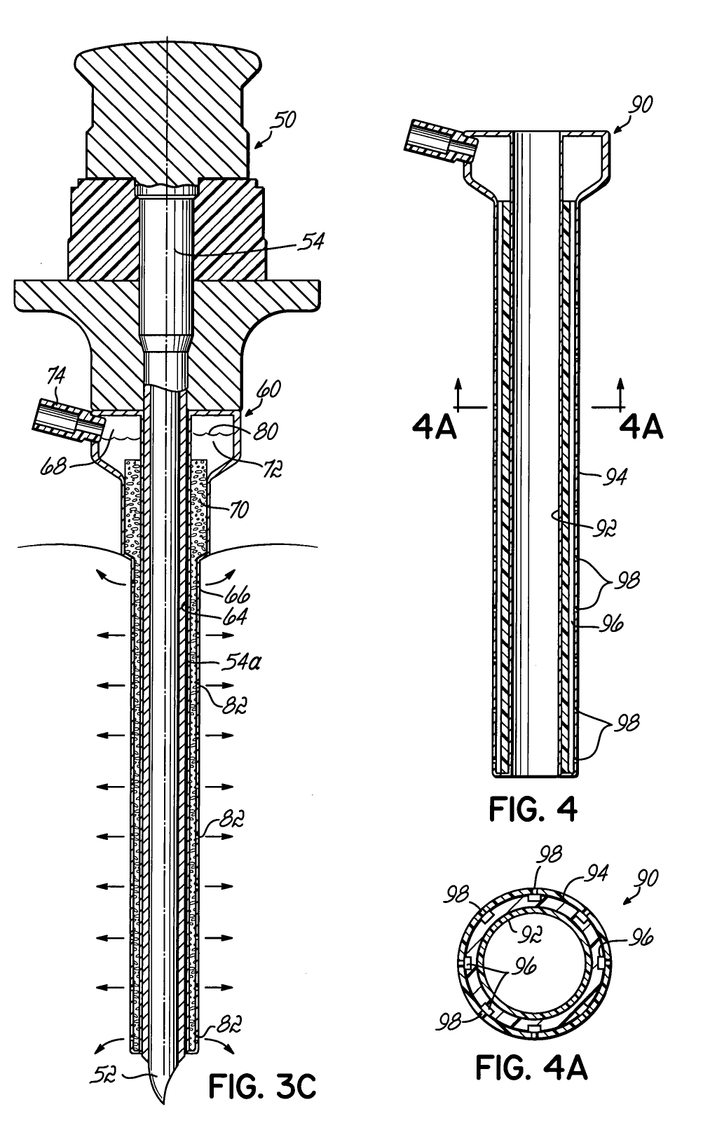

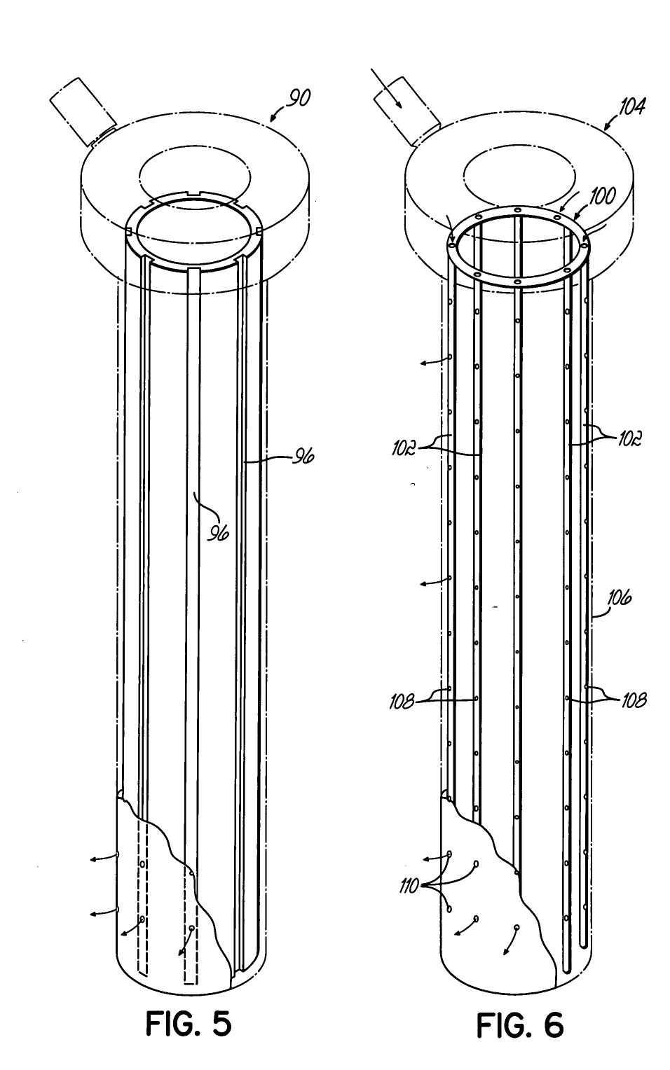

FIGS. 4, 4A and 5 illustrate another embodiment of a fluid delivery device 90 respectively in longitudinal cross section, and transverse cross section. This device 90 is similar to the device described with respect to FIGS. 3A-3C, with the exceptions being that no sponge or fluid absorbent material is utilized, and the two layers or walls 92, 94 of the fluid delivery device 90 are not separated by a cylindrical, annular void space but instead are separated by channels 96 extending along the length of the device 90 and communicating with respective perforations 98 in the outer layer 94. In this embodiment, the channels 96 are shown as being formed on the outer surface of the inner layer 92, however, it will be understood that the channels 96 may be formed on the inner surface of the outer layer 94, or on both the inner and outer layers 92, 94. In this embodiment as well, the outer layer 94 may not need to be flexible as the fluid is forced out of the perforations 98 as opposed to being squeezed out of the perforations during insertion. The forceful ejection of fluid may take place by the pressure developed, for example, by the pump mechanism such as a syringe (not shown) being used to introduce the fluid.

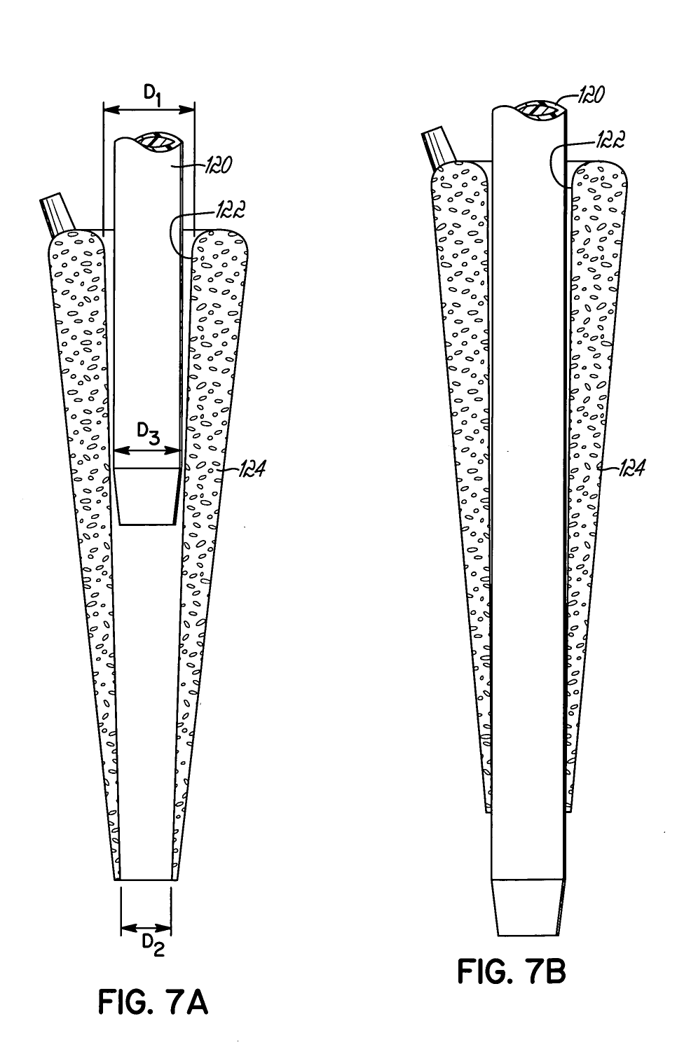

FIG. 6 is a perspective view similar to FIG. 5, but illustrating another embodiment in which a skeletal structure 100 of elongate tubes 102 is used in place of channels on the outside of an inner layer of the fluid delivery device. In this embodiment, the fluid delivery device 104 may have inner and outer layers with the skeletal structure 100 of perforated tubes 102 sandwiched between the inner and outer layers. Only an outer layer 106 is shown. The perforations 108 of the tubes 102 may align with perforations 110 in the outer layer 106, or the outer layer 106 may be, for example, formed from a more generally perforate structure to allow transfer of the fluid to the outer surface thereof and into the tissue of the patient.

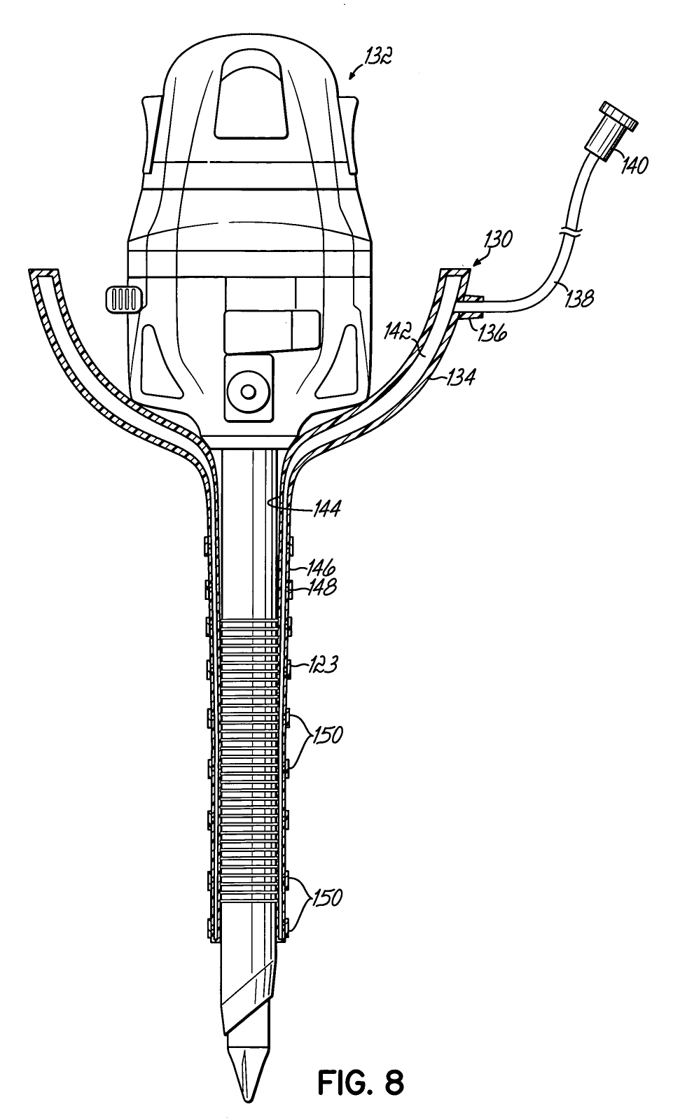

FIGS. 7A and 7B illustrate partially sectioned side views of an embodiment which is similar to the embodiment discussed above with respect to FIGS. 2A and 2B. FIGS. 7A and 7B illustrate various dimensional relationships appropriate for allowing easy insertion, but adequate retention of the fluid delivery device on the outside of the cannula 120 of a trocar-cannula complex. In this regard, the diameter D₁ of the internal bore 122 at the proximal end of the fluid delivery device 124 is greater than the inner diameter D₂ at the distal end thereof. In addition, D₁ is greater than the outer diameter D₃ of cannula 120 and D₂ is less than D₃. Therefore, the cannula 120 will be easily inserted at the proximal end of the fluid delivery device 124, yet securely retained by friction at the distal end as illustrated in FIG. 7B.

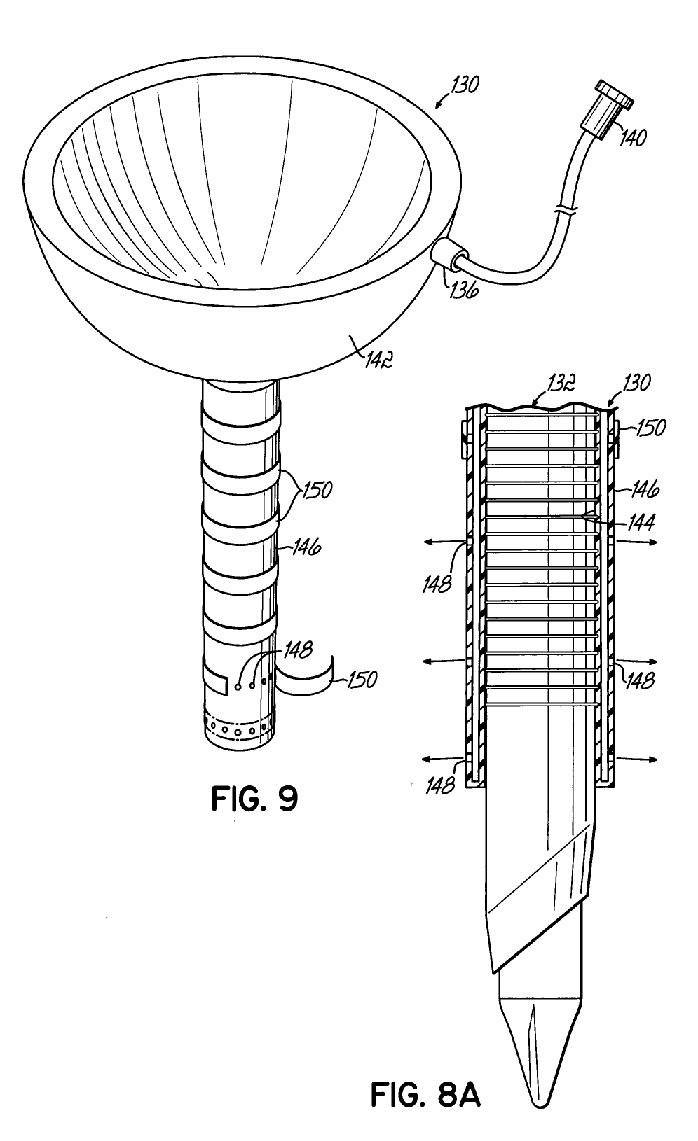

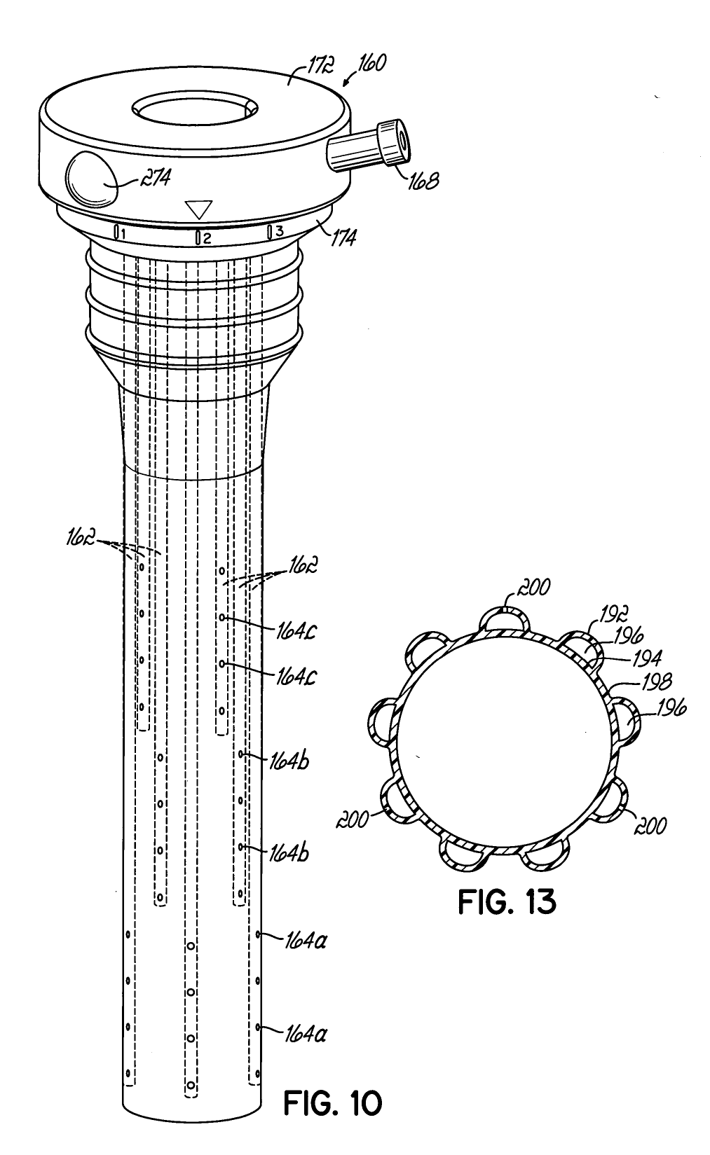

FIGS. 8, 8A and 9 illustrate yet another embodiment of the invention in the form of a fluid delivery device 130 which, like the previously discussed embodiments, is universally attachable to any existing trocar-cannula complex 132, such as that shown in FIG. 8, and which includes an upper generally cup-shaped reservoir 134 for receiving the biologically active liquid from an inlet 136 coupled with, for example, a fluid delivery line 138 and a suitable connector 140 for a pump mechanism, such as a standard syringe (not shown). The biologically active liquid will enter the upper cup-shaped reservoir 134 in an inner portion 142 of the fluid delivery device 130 and travel by gravity, or forceful pressure, or both, to the distal end of the device 130 between inner and outer layers 144, 146, as best shown in FIG. 8A. In this embodiment, the outer layer 146 includes perforations 148 which are covered by a removable tape 150 so that the surgeon may selectively expose the perforations 148 as illustrated in FIG. 9 for accurate delivery of the fluid into the tissue of the patient as indicated by the arrows in FIG. 8A. For example, for some thinner patients, perforations 148 at a more distal end of the device 130 may be exposed, while for heavier patients, perforations 148 closer to the proximal end of the device 130 may be additionally or alternatively exposed. This allows delivery of the fluid as schematically illustrated in FIG. 8A to the desired tissue in an accurate manner.

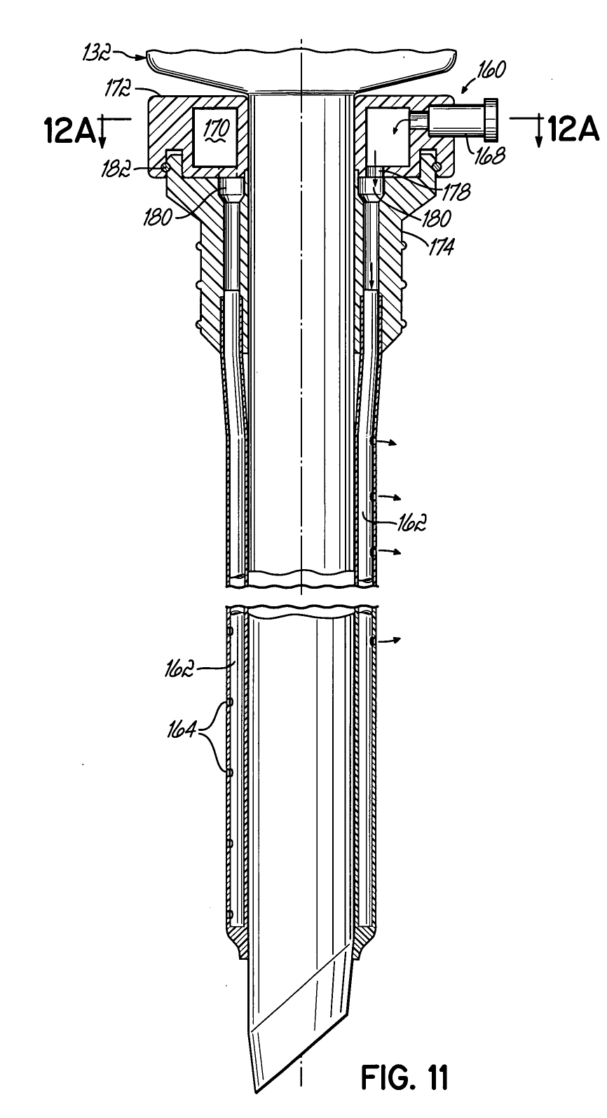

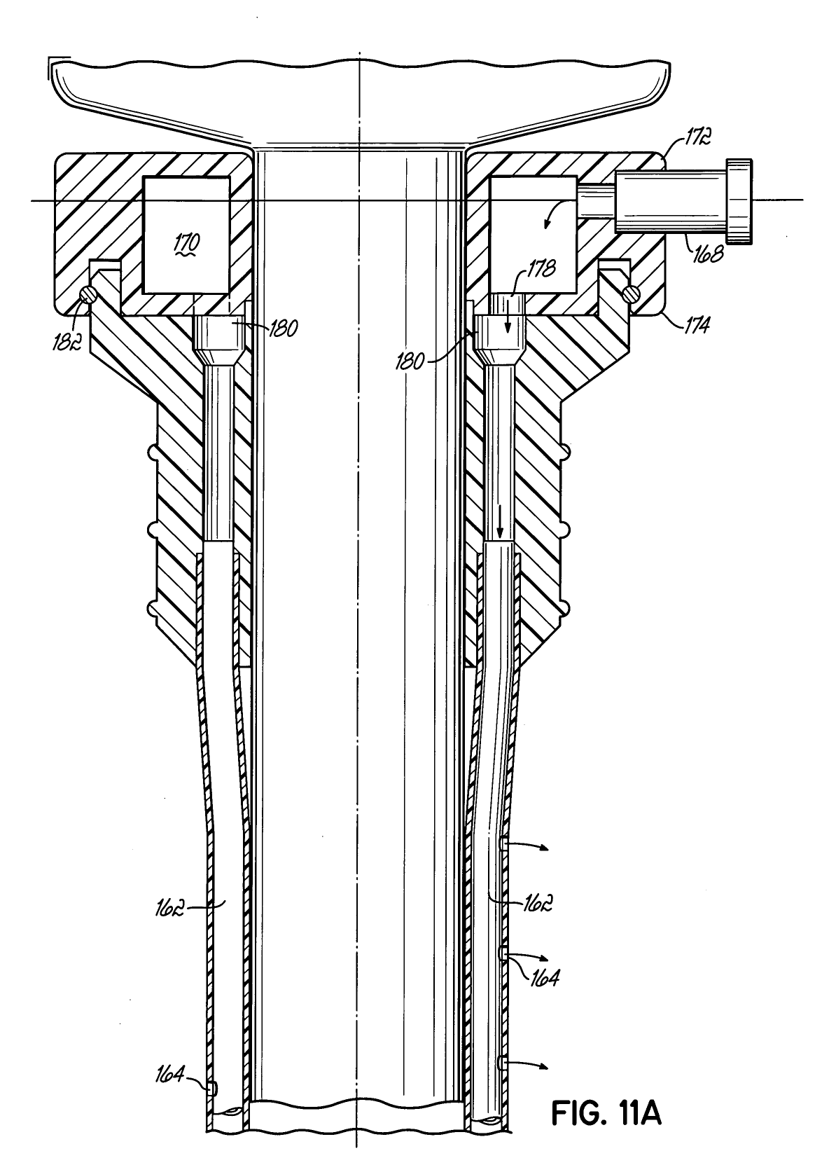

FIGS. 10, 11, 11 A and 12A-12B illustrate another embodiment of the invention in the form of a fluid delivery device 160 which again may be universally and removably coupled to any trocar-cannula complex. As shown best in FIG. 10, the fluid delivery device 160 includes internal channels 162 which may be formed in various manners, including but not limited to, the manners discussed previously. These channels 162 are formed with perforations 164 in an outer layer which extend along different lengths of the device 160. In the embodiment shown, three different lengths of channels 162 are illustrated, but it will be understood that a greater or fewer number of selections may be provided in the device 160. In addition, in the illustrative embodiment each of the different length channels 162 becomes shorter in a direction extending toward the proximal end of the device 160. It will also be appreciated that the different channels 162 may instead have perforations along any desired incremental length of the fluid delivery device. For example, as shown FIG. 10, one channel 162 or subset of channels 162 may have perforations 164a at only the distal end, a second channel 162 or subset of channels 162 may have perforations 164b only at an intermediate portion of the length, and a third channel 162 or subset of channels 162 may have perforations 164c only at a proximal portion of the length. Upper component 172 may be rotated or dialed relative to lower component 174 to select indicia such as “1”, “2”, or “3”, as shown, depending on which set of perforations 164a, 164b, or 164c the surgeon desires to dispense fluid through in accordance with the invention. Additional, corresponding indicia (not shown) may be placed on the outer surface of the device so that, for example, the number “1”, “2”, or “3” will be visible to the surgeon just above the patient's skin at the port site thereby indicated the appropriate number on the dial to select for accurate fluid delivery.

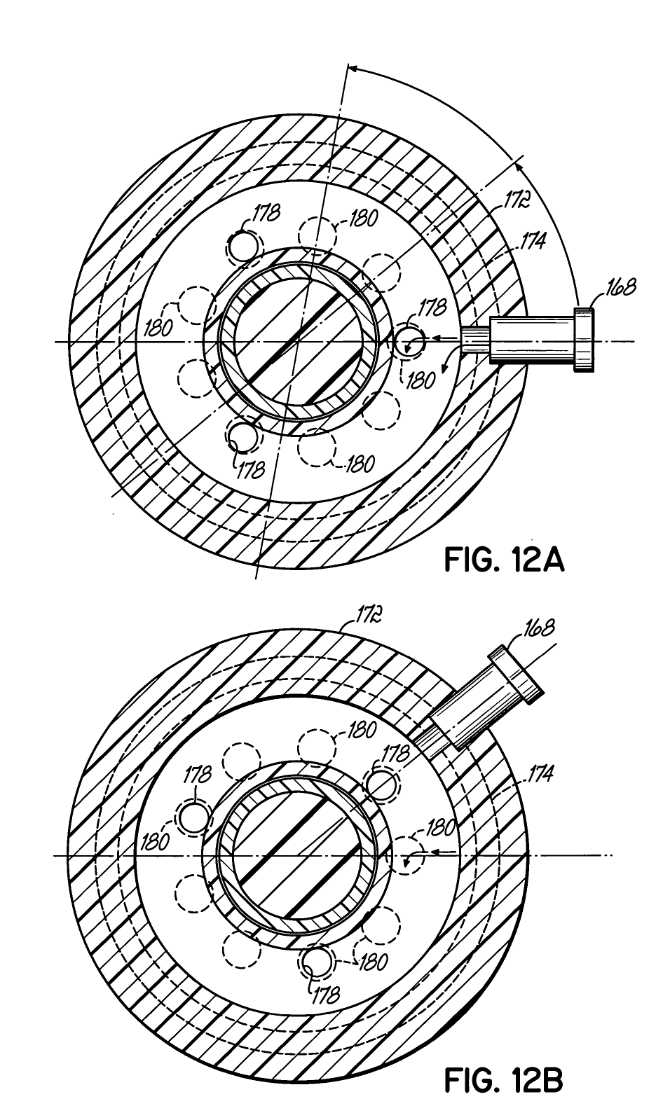

As shown in FIGS. 11 and 11A, fluid is introduced into an inlet 168, such as in a manner previously described, and is delivered into a proximal chamber or void space 170 which is in fluid communication, selectively, with the respective channels 162 contained along the length of the device 160. The upper or proximal portion of the device 160 is formed in at least two components 172, 174 which are movable and, preferably, rotatable with respect to each other to selectively align fluid delivery passages 178, 180 between the chamber 170 and the selected subset of channels, based on the desired delivery location for the fluid along the length of the device 160. An O-ring 182 or other appropriate seal may be used between the two rotatable components 172, 174 of the fluid delivery device 160. By further review of FIGS. 12A and 12B, it will be appreciated that rotating the upper component 172 relative to the lower component 174 will align the fluid delivery passages 178 in the upper component 172 with a selected group of fluid delivery passages 180 in the lower component 174 which then communicate with a corresponding group of fluid delivery channels 162 and perforations 164 along the desired length of the fluid delivery device 160. This allows the surgeon to easily select the location along the length of the fluid delivery device 160 to dispense the biologically active liquid into the tissue (not shown).

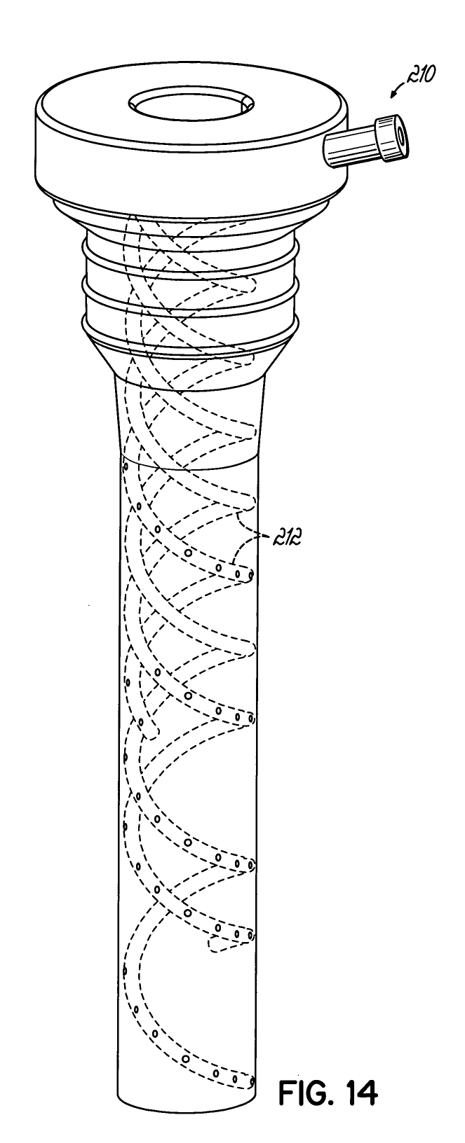

FIG. 13 illustrates a transverse cross section of a fluid delivery device 140 formed in another alternative manner. That is, an outer layer 192 is secured, such as through ultrasonic welding, to an inner layer 194 in such a manner that a plurality of longitudinally extending channels 196 are formed between the weld locations 198. The channels 196, as previously described include perforations 200 along the same or different selected length portions of the fluid delivery device 190.

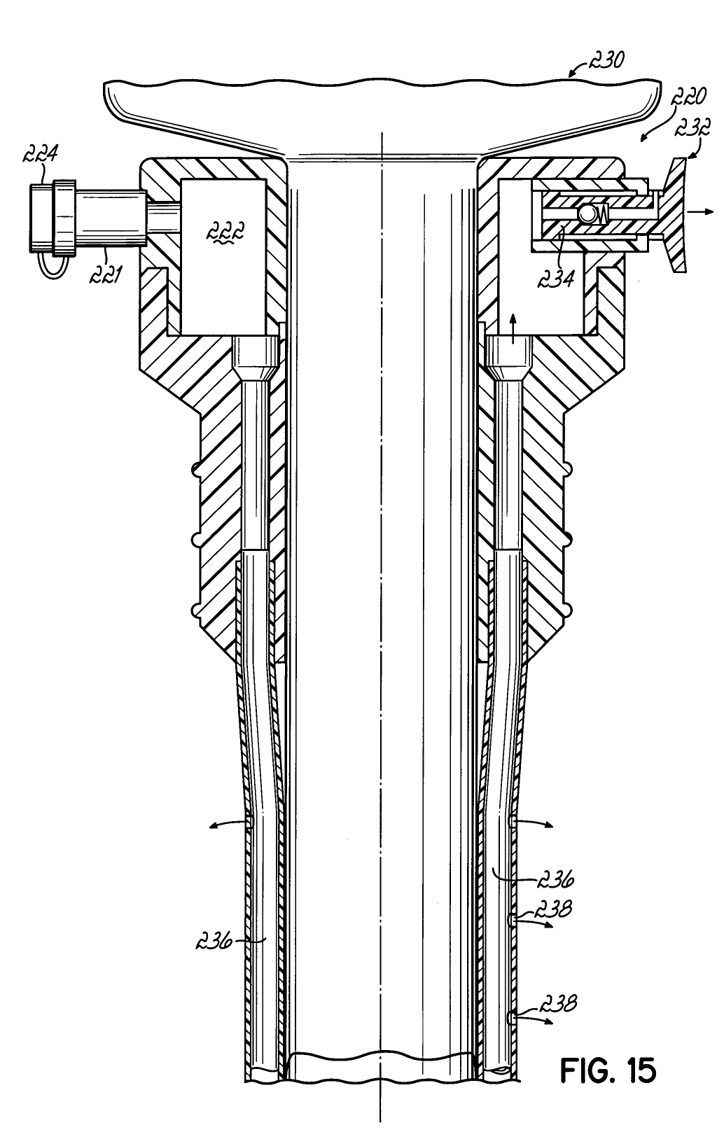

FIG. 14 is a perspective view illustrating another alternative fluid delivery device 210 which is the same as that shown in FIG. 10, except that the channels 212 are formed in a spiral or helical fashion along the length of the fluid delivery device 210. It will be understood that other configurations of fluid delivery channels may be used as well.

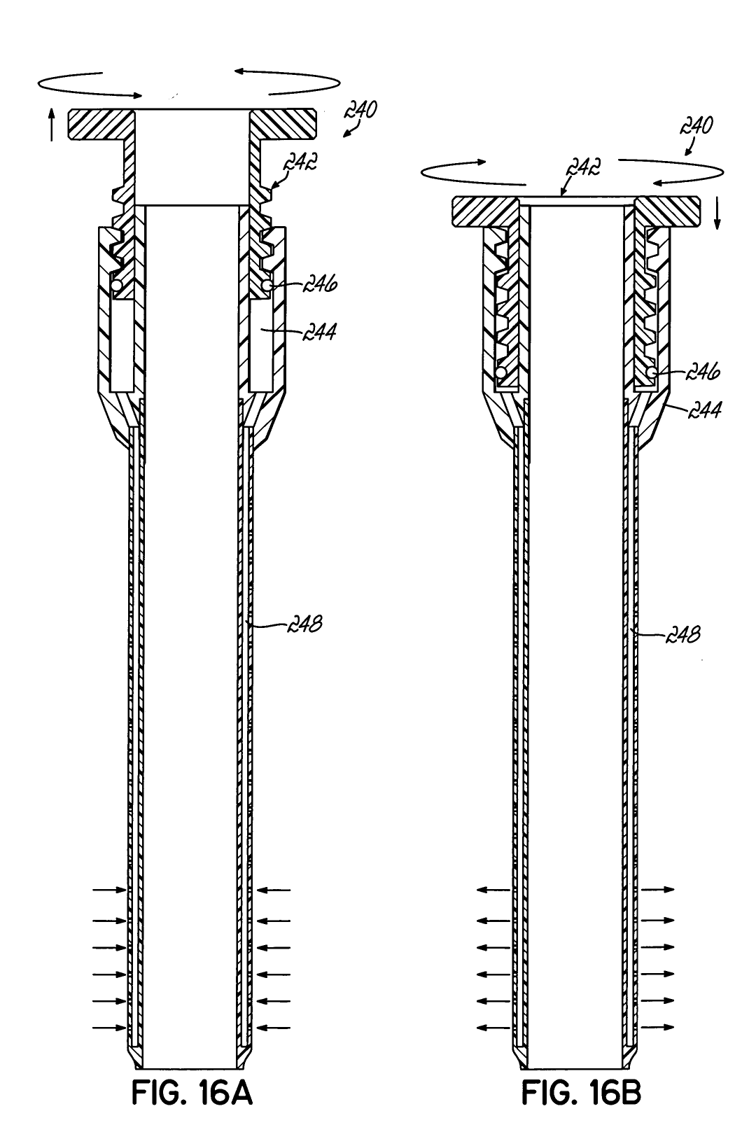

FIG. 15 illustrates a cross sectional view similar to FIG. 11A, but illustrating an alternative fluid delivery device 220. Device 220 includes a connector 221, for example, adapted to be connected to a syringe (not shown) for filling the void space or chamber 222 through an inlet. This connector may then be sealed by way of a cap 224. After insertion of the trocar-cannula complex 230 and the connected fluid delivery device 220 into a port site of a patient, the biologically active liquid is forced into the patient's tissue by another integrated pump mechanism 232 which may be similar to a priming pump mechanism and includes a piston member 234 which pressurizes the inner chamber 222 and the selectively, fluidly coupled channels 236 to thereby force the liquid from the perforations 238. If the liquid is depleted from the chamber 222 and the surgeon desires to introduce additional liquid, a syringe may be used to again fill the chamber 222 through the connector 221. All other aspects of this embodiment may be similar or the same as discussed with respect to the embodiment of FIG. 11A.

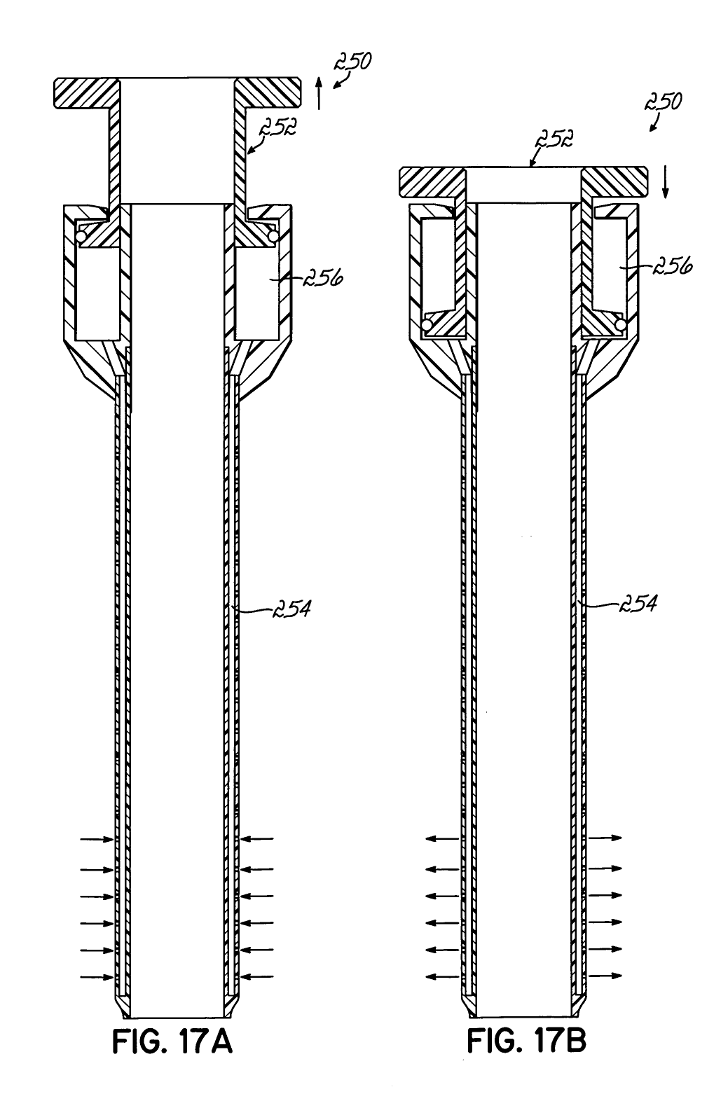

FIGS. 16A and 16B illustrate side cross sectional views of yet another embodiment of a fluid delivery device 240 which is similar to those embodiments discussed above, except that a screw pump mechanism 242 is illustrated for drawing liquid into the device 240, or expelling liquid from the device, or both. As shown in FIG. 16A, a screw pump-type mechanism 242 is located in a proximal chamber 244 and includes, for example, an O-ring 246 for sealing purposes. The screw mechanism 242 may be rotated in a proximal direction to initially draw fluid into an inner portion 248, such as channels or an annular space, formed in the fluid delivery device 240, as well as into the proximal chamber 244 during an initial liquid filling phase and prior to insertion of the device 240 with a trocar-cannula complex (not shown) into a patient. Once the fluid delivery device 240 is filled with the desired liquid in this manner, or another manner, the screw mechanism 242 may be rotated in a distal direction as shown in FIG. 16B (and after insertion into the patient) to force the liquid into the tissue of the patient.

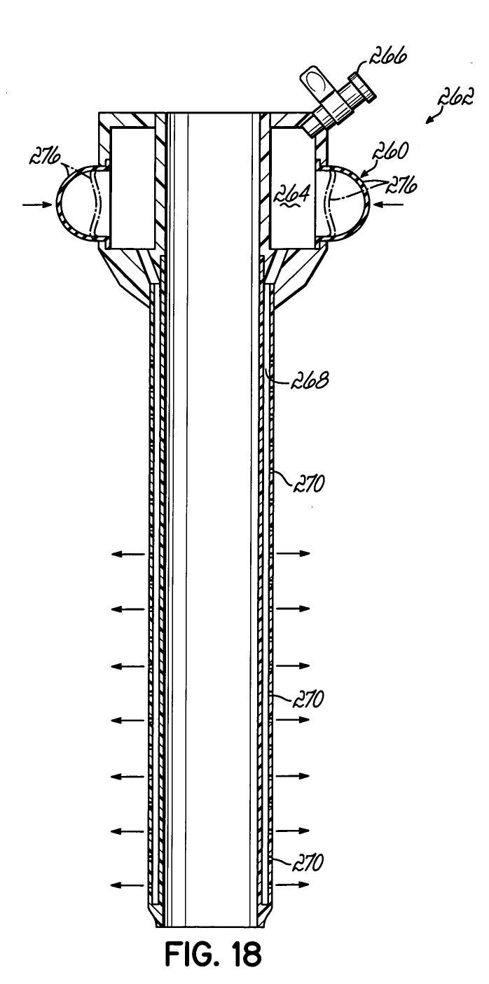

FIGS. 17A and 17B are illustrations of another fluid delivery device 250 constructed in accordance with an embodiment similar to that shown in FIGS. 16A and 16B, but illustrating a more standard piston-type pump mechanism 252 as opposed to a screw pump-type mechanism. In this embodiment, the piston pump 252 may be axially withdrawn in a proximal manner as shown in FIG. 17A to fill the inner portion 254, such as an annular void space or series of channels, as well as a proximal chamber 256. The piston pump 252 may then be axially depressed in a distal direction as shown in FIG. 17B to force the desired biologically active liquid into the desired tissue of a patient, such as in one of the manners described herein.

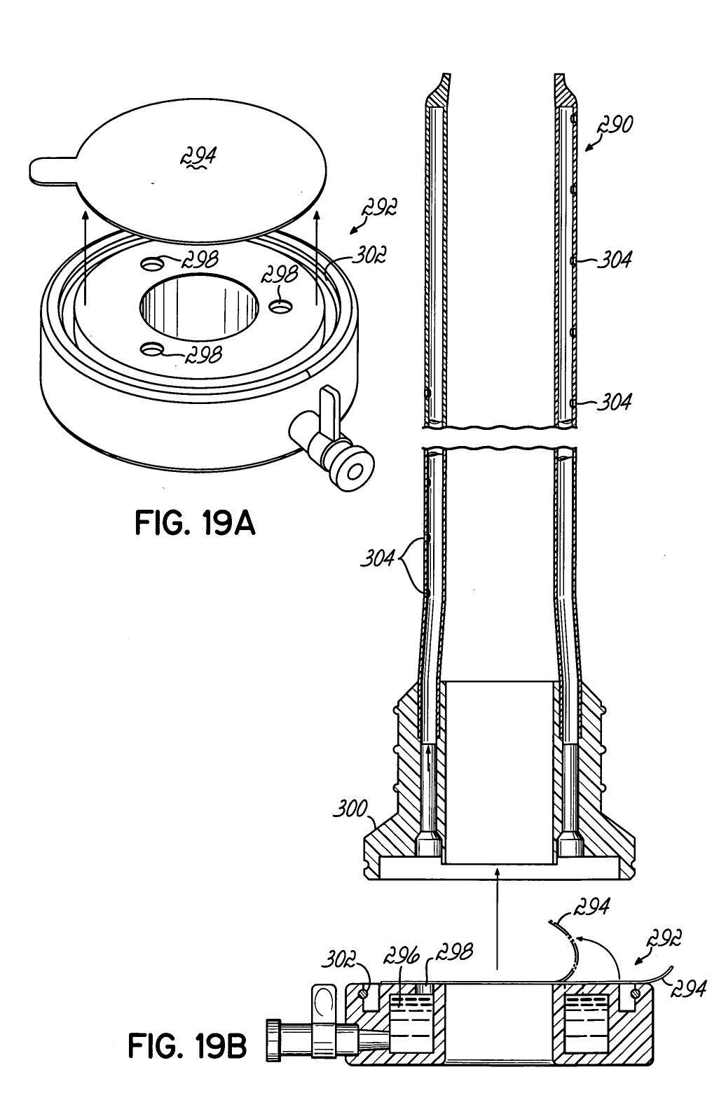

FIG. 18 illustrates a longitudinal cross sectional view similar to the embodiments of FIGS. 16A, 16B, 17A and 17B, but illustrating yet another pumping mechanism 260 for forcing biologically active liquid from a selected portion of a fluid delivery device 262. In this embodiment, the proximal chamber portion 264 of the device 262 includes a connector 266 for coupling with a suitable filling mechanism such as a syringe (not shown) for filling the interior chamber or void space 264 with the desired biologically active liquid. This chamber 264 communicates with an inner portion, such as a series of channels, or an annular void space 268 (with or without a fluid absorbent material, not shown) for delivery of the liquid to the tissue of the patient. For forcing the liquid from perforations 270 in the outer layer of the device two bulbous flexible membranes 274, 276 are provided and may be depressed by a user. This forces liquid from the upper chamber or void space 264 through delivery passages 280 and into the inner portion 268, such as the void space or channels, and out of the perforations 270. This is illustrated by the arrows and by illustrating the depressed configuration of the membranes or flexible members 274, 276 in dash-dot lines.

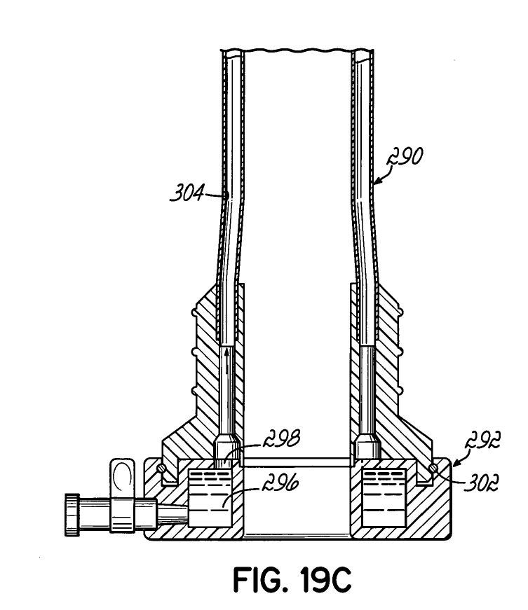

FIGS. 19A-19C illustrate yet another embodiment of a fluid delivery device 290 constructed in accordance with the invention. This device 290 may be formed, for example, as discussed in connection with the embodiment of FIGS. 11, 11A, 12A and 12B. The difference in the embodiment of FIGS. 19A-19C is that a prefilled proximal component 292 of the fluid delivery device 290 is provided so that the device 290 is readily usable by the surgeon. That is, device 290 can require less preparation time by the surgery team. In this regard, a removable seal 294, as illustrated in FIGS. 19A and 19B is provided to initially contain the biologically active liquid in the proximal chamber or void space 296 of the proximal component 292. The seal 294 is removed as shown in FIG. 19B with the device 290 inverted (i.e., the fluid delivery passages 298 are directed upwardly as shown) and the distal component 300 of the device 290 is snapped into place and retained, for example, by an O-ring 302, and/or any other suitable connector/seal assembly. The assembled device 290 is shown in FIG. 19C. An integrated pump mechanism (not shown), such as previously described with respect to FIG. 15, FIGS. 16A-B, FIGS. 17A-B, or FIG. 18, may be used to force the liquid through the device 290 and out of the selected perforations 304. It will further be understood that the proximal component 292 illustrated in FIGS. 19B and 19C may rotate with respect to the attached distal component 300 for selection of the desired fluid delivery location as described with respect to the embodiment of FIGS. 11, 11A, 12A and 12B.

Many different types of irrigation fluids may be introduced through the fluid delivery devices of this invention. These include, but are not limited to, saline solutions, lidocaine-containing fluids, betadine-containing fluids, cancer treatment fluids, or any other fluid or pharmaceutically acceptable formulation necessary or desired for a particular medical procedure. In addition, fluids other than irrigation fluids or treatment fluids may be delivered through the devices of this invention. As one additional example, bioadhesives may be delivered to an incision site or any other necessary tissue repair site to provide for quicker and more effective administration of the adhesive to the desired site.

These fluids are pharmaceutically acceptable formulations that contain biologically active agents that the surgeon can infuse to the port site and intervening tissue layers. Examples of active agents include, but are not limited to various types of anesthetics, therapeutic peptides, polypeptides, macromolecules such as proteins (e.g., monoclonal antibodies), oligonucleotides (e.g., antisensenucleotides), lipid components, lipid formulations, liposome substances, immunoglobins, immunomodulators, steroids, antiangiogenic agents, cancer chemotherapeutic agents, anti-infectives (antibiotics, antiviral, etc.), cytotoxins, anticoagulants, fibrinolytic agents, anti-inflammatory agents and combinations thereof.

The pharmaceutically acceptable formulations as known to one skilled in the art may contain the biologically active agents in a freely soluble form for immediate effect at the tissue site or in a controlled or sustained release matrix for a long-term effect such as hours or days, or a combination of both.

The controlled or sustained released matrix may be biologically degradable and prepared using procedures as known to one skilled in the art. The form of the matrix may be selected, for example, from microporous films, microspheres, nanospheres, micelles, liposomes, powders, microparticles, and hydrogels. These matrices may be a component of the pharmaceutically acceptable formulation that is delivered to the port. They then diffuse into the surrounding tissue and become embedded or implanted in the tissue. Thus, they impart a sustained effect of the active agent due to its controlled release from the matrix as it degrades.

Biologically degradable matrices may be formed by procedures known to one skilled in the art. For example, such components may be various types of lipids that form micelles and liposomes, polymers and copolymers of polyorthoesters, polyethylene glycol, ketene acetals, polyols and others. Examples of the various biodegradable polymers, various biologically active agents that become entrapped or encapsulated in the formed matrices as previously described, injectable fluid dosage forms, and semi-solid pharmaceutical compositions are described in U.S. Pat. No. 6,524,606; U.S. Pat. No. 6,667,371; U.S. Pat. No. 6,613,355; U.S. Pat. No. 5,968,543; U.S. Pat. No. 5,939,453; U.S. Pat. No. 4,957,998; U.S. Pat. No. 4,946,931; U.S. Pat. No. 4,855,132; U.S. Pat. No. 4,764,364; U.S. Pat. No. 4,304,767; and U.S. Published Applications 2002/0037300, 2003/0130472, 2002/0168336, 2002/0176844, and 2003/0212148, the disclosures of which are incorporated herein in their entirety. Other dosage forms and biologically active agents in pharmaceutically acceptable formulations may be used as well.

Many different types of trocars and cannulas may be utilized within the scope of this invention. These trocars and cannulas may be inserted through a port site of a patient together or separately, for example, by using a needle introducer for an expandable cannula and subsequently introducing the cannula of the trocar-cannula complex. The cannula is configured to receive laparoscopic and arthroscopic instruments, and other instruments used during minimally invasive surgery.

The use of the invention eliminates or at least reduces the handling of needles during the surgical procedure and the trocar-cannula assembly or complex allows accurate delivery to the port site. The active agent is delivered to the port site fast and simple. Both short and long acting active agents may be delivered to ameliorate various biological responses such as the pain cascade in a physiological fashion. The assembly also allows the surgeon to choose what to infuse or irrigate for any particular case and may be infused at any time during the procedure and as many times as is necessary such as after the initial introduction of the assembly through the port site, during the surgical procedure, or at the end of the procedure.

While the present invention has been illustrated by a description of a preferred embodiment and while this embodiment has been described in some detail, it is not the intention of the Applicant to restrict or in any way limit the scope of the appended claims to such detail. Additional advantages and modifications will readily appear to those skilled in the art. The various features of the invention may be used alone or in numerous combinations depending on the needs and preferences of the user. This has been a description of the present invention, along with the preferred methods of practicing the present invention as currently known. However, the invention itself should only be defined by the appended claims, wherein I claim:

1. A medical apparatus for dispensing a biologically active liquid on the outside of a cannula, comprising:
a fluid delivery device configured to be removably coupled to the cannula, said fluid delivery device including an inner portion adapted to receive the biologically active liquid, an outer surface adapted to contact the tissue of the patient, and at least one pathway from the inner portion to the outer surface for delivering the biologically active liquid from the inner portion to the outer surface when the outer surface is in contact with the tissue.

2. The apparatus of claim 1, wherein said inner portion further comprises a sponge material.

3. The apparatus of claim 1, wherein said inner portion further comprises a void space.

4. The apparatus of claim 1, wherein said inner portion further comprises a plurality of discrete void spaces.

5. The apparatus of claim 4, wherein said plurality of discrete void spaces further comprise a plurality of fluid delivery channels extending generally along the length of the cannula.

6. The apparatus of claim 5, wherein said fluid delivery channels are parallel to the length of the cannula.

7. The apparatus of claim 5, wherein said fluid delivery channels spiral along the length of the cannula.

8. The apparatus of claim 1, wherein said fluid delivery device is frictionally engaged with said cannula.

9. The apparatus of claim 1, further comprising:
an actuator coupled to said fluid delivery device and configured to force the biologically active liquid along said pathway to said outer surface.

10. The apparatus of claim 9, wherein said actuator further comprises a syringe.

11. The apparatus of claim 9, wherein said actuator further comprises a pump mechanism.

12. The apparatus of claim 11, wherein said pump mechanism further comprises a movable piston member operative to displace fluid from said inner portion.

13. The apparatus of claim 11, wherein said pump mechanism further comprises a flexible member adapted to be depressed by a user to displace fluid from said inner portion.

14. The apparatus of claim 1, wherein said fluid delivery device including a length, and further comprising:
a fluid path selection device operatively coupled with said fluid delivery device for selectively allowing the biologically active liquid to be dispensed along different portions of the length of said fluid delivery device.

15. The apparatus of claim 14, wherein said inner portion is divided into at least first and second inner portions, said first inner portion extending along a greater portion of the length of said cannula than said second inner portion, and said fluid path selection device is capable of being selectively actuated to deliver the biologically active liquid from one of the first and second inner portions.

16. The apparatus of claim 1, wherein said fluid delivery device further comprises a proximal, preloaded chamber of said biologically active liquid, and a distal fluid delivery section having said inner portion and said outer surface.

17. The apparatus of claim 16, wherein said preloaded chamber is removably coupled to said fluid delivery section.

18. A medical apparatus for dispensing a biologically active liquid, comprising:
a trocar-cannula complex configured to be inserted through selected tissue of a patient and including a cannula and an elongated trocar having a closed converging tip at a distal end thereof and a fluid delivery channel, said cannula having a working channel defined therein through which surgical instruments may be advanced, and
a fluid delivery device removably coupled to said cannula, said fluid delivery device including an inner portion adapted to receive the biologically active liquid, an outer surface adapted to contact the tissue of the patient, and at least one pathway from the inner portion to the outer surface for delivering the biologically active liquid from the inner portion to the outer surface when the outer surface is in contact with the tissue.

19. The apparatus of claim 18, wherein said inner portion further comprises a sponge material.

20. The apparatus of claim 18, wherein said inner portion further comprises a void space.

21. The apparatus of claim 18, wherein said inner portion further comprises a plurality of discrete void spaces.

22. The apparatus of claim 21, wherein said plurality of discrete void spaces further comprise a plurality of fluid delivery channels extending generally along the length of the cannula.

23. The apparatus of claim 22, wherein said fluid delivery channels are parallel to the length of the cannula.

24. The apparatus of claim 22, wherein said fluid delivery channels spiral along the length of the cannula.

25. The apparatus of claim 18, wherein said fluid delivery device is frictionally engaged with said cannula.

26. The apparatus of claim 18, further comprising:
an actuator coupled to said fluid delivery device and configured to force the biologically active liquid along said pathway to said outer surface.

27. The apparatus of claim 26, wherein said actuator further comprises a syringe.

28. The apparatus of claim 26, wherein said actuator further comprises a pump mechanism.

29. The apparatus of claim 28, wherein said pump mechanism further comprises a movable piston member operative to displace fluid from said inner portion.

30. The apparatus of claim 28, wherein said pump mechanism further comprises a flexible member adapted to be depressed by a user to displace fluid from said inner portion.

31. The apparatus of claim 18, further comprising:
a fluid path selection device operatively coupled to said first and second portions for selectively allowing the biologically active liquid to be dispensed along different length portions of said cannula.

32. The apparatus of claim 31, wherein said inner portion is divided into at least first and second inner portions, said first inner portion extending along a greater portion of the length of said cannula than said second inner portion, and said fluid path selection device is capable of being selectively actuated to deliver the biologically active liquid from one of the first and second inner portions.

33. The apparatus of claim 18, wherein said fluid delivery device further comprises a proximal, preloaded chamber of said biologically active liquid, and a distal fluid delivery section having said inner portion and said outer surface.

34. The apparatus of claim 33, wherein said preloaded chamber is removably coupled to said fluid delivery section.

35. A method of performing a minimally invasive surgical procedure using a trocar-cannula complex with a cannula portion having a lumen configured to receive a trocar, the method comprising:
affixing a removable fluid delivery device to the cannula portion, the fluid delivery device having a fluid passage configured to receive fluid and deliver the fluid to an outside surface of the cannula portion
introducing the trocar-cannula complex and the fluid delivery device through a port site of a patient, and
delivering the fluid from the fluid passage to the outside surface of the fluid delivery device into contact with the patient.

36. The method of claim 35, wherein the fluid is an irrigant.

37. The method of claim 35, wherein the fluid includes a pain medication.

38. The method of claim 35, wherein the fluid is a tissue adhesive.

39. The method of claim 35, wherein the fluid is delivered to the fluid passage by a pump.

40. The method of claim 35, wherein the fluid is delivered to the inlet by a syringe.
